(19) United States
(12) Patent Application Publication
(10) Pub. No.: US 2010/0305438 A1
(43) Pub. Date: Dec. 2, 2010

(54) SYSTEM AND METHOD FOR SCALING STRAIN IMAGE DATA

(76) Inventor: Kenneth Wayne Rigby, Clifton Park, NY (US)

Correspondence Address: GENERAL ELECTRIC COMPANY;GLOBAL RESEARCH, ONE RESEARCH CIRCLE, BLDG. K1-3A59, NISKAYUNA, NY, 12309, US

(21) Appl. No.: 12/475,191

(22) Filed: May 29, 2009

(51) Int. Cl. A61B 8/00; A61B 5/103

(52) U.S. Cl. 600/438; 600/587

(57) ABSTRACT

A computer readable storage medium has a computer program stored thereon, which includes a set of instructions that when executed by a computer causes the computer to access positional data acquired from a material at a plurality of states of compression. The set of instructions also causes the computer to estimate a plurality of strain data sets from the positional data, each of the plurality of strain data sets corresponding to a different state of compression, and compare a first pair of strain data sets of the plurality of strain data sets with each other. The set of set of instructions further causes the computer to calculate a first measure of correlation from the comparison, scale a first strain data set of the first pair of strain data sets if the first measure of correlation is above a correlation threshold, and display the scaled first strain data set to a user.

### BACKGROUND OF THE INVENTION

The present invention relates generally to the use of ultrasound to study soft biological tissue and, more particularly, to the measurement of elastic properties of the tissue.

Determining the mechanical properties of biological tissue (e.g., parameters of elasticity) is of fundamental interest in clinical diagnosis because of the correlation between the healthy or pathological state of a tissue and its stiffness. It is known that some cancers are stiffer than normal tissues. This is the basis for hand palpitation used by physicians to diagnosis these disorders as well as breast and testicular self examinations. Beyond these more rudimentary techniques, imaging modalities capable of determining the relative stiffness of various tissues can also be very beneficial to cancer diagnosis in soft biological tissue.

In recent years, ultrasound has been used to detect spatial variations in the elastic properties of biological tissue. This capability has led to a new imaging technique known as ultrasound elasticity imaging or ultrasound strain imaging. In ultrasound strain imaging, ultrasound data is used to create an image of the displacement or strain profile, which is related to elasticity within a region of interest (ROI). The resulting image of the strain profile often reveals structures that may be invisible or hard to detect in a traditional B-mode image.

Because strain is a function of the change in position, at least two frames of data containing tissue position information are used for each estimate of strain in ultrasound strain imaging. In one method, an operator generates the data used to estimate strain by manipulating the ultrasound transducer in a roughly sinusoidal push-pull motion on a subject's skin. The movement of the transducer varies the stress in the patient's tissue by cyclically compressing and decompressing the tissue. The displacement of tissue caused by the transducer is estimated by comparing frames of differing (and unknown) stress levels. Strain may then be estimated from the estimated tissue displacement.

One difficulty with the ultrasound strain imaging method is that strain itself is not a property of the tissue, but varies with the applied stress, which varies continuously as the transducer moves. The elastic modulus, the ratio of strain to stress, is a property of the tissue. The observed strain can often be a useful substitute for the elastic modulus if some way of removing this varying scale factor, or reducing its effect, can be found. A second difficulty is that the tissue displacement may be very small between some frames. For example, at the end of each push and pull stroke, there is little to no change in the displacement between adjacent B-mode frames. Because the displacement is equal to or near zero, the estimated strain data and resulting image based on these adjacent frames will likely include a significant amount of noise and/or artifact. Further, variations in the angle, location, and the pressure with which the transducer is applied may affect the scale of the estimated strain data and/or the noise present therein, thus producing useless or “bad” data for some frames. The issues and artifacts just described are also present in strain estimation systems that do not rely on manual techniques for inducing tissue displacements. These issues and artifacts are also present in imaging methods other than ultrasound which may be used for estimating strain, such as magnetic resonance imaging.

One known technique for improving the usefulness of the display of estimated strain data involves normalizing the measured strain over a selected portion of the frame. Such a technique may calculate the average strain in the center region of interest (ROI) of the frame, for example, and then divide all of the strain data in the frame by the calculated average strain to scale the strain data. However, such a technique does not account for inconsistencies in the user's manipulation of the transducer or the potential noise and/or artifacts resulting from strain data at the end of each push and pull stroke.

Thus, there still is a need for improving the usefulness of the display of the measured strain, and reducing the noise and artifacts in the displayed strain image.

It would therefore be desirable to have a system and method that displays strain while accounting for the frame-to-frame variability and uncertainty in induced tissue stress.

### BRIEF DESCRIPTION OF THE INVENTION

The present invention is directed to a system and method for scaling strain image data.

Therefore, in accordance with one aspect of the present invention, a computer readable storage medium has a computer program stored thereon, which includes a set of instructions that when executed by a computer causes the computer to access positional data acquired from a material at a plurality of states of compression. The set of instructions also causes the computer to estimate a plurality of strain data sets from the positional data, each of the plurality of strain data sets corresponding to a different state of compression, and compare a first pair of strain data sets of the plurality of strain data sets with each other. The set of set of instructions further causes the computer to calculate a first measure of correlation from the comparison, scale a first strain data set of the first pair of strain data sets if the first measure of correlation is above a correlation threshold, and display the scaled first strain data set to a user.

In accordance with another aspect of the present invention, a method includes accessing a first set of positional data, accessing a second set of positional data, and estimating a first set of strain data from the first set of positional data. The method also includes estimating a second set of strain data from the second set of positional data and comparing the first set of strain data with the second set of strain data. The method further includes calculating a first measure of correlation from the comparison, scaling the first set of strain data if the first measure of correlation is above a correlation threshold, and displaying the scaled first set of strain data to a user.

In accordance with yet another aspect of the present invention, a system includes an imaging device configured to acquire a plurality of positional data sets, each positional data set comprising positional data of a material at a respective state of compression and a computer. The computer includes one or more processors programmed to access the plurality of positional data sets, estimate a first set of strain data from a first positional data set of the plurality of positional data sets, and estimate a second set of strain data from a second positional data set of the plurality of positional data sets. The one or more processors are further programmed to compare the first set of strain data with the second set of strain data, determine a first measure of correlation based on the comparison, scale the first set of strain data if the first measure of correlation is above a correlation threshold, and display an image of the scaled first set of strain data on a display.

Various other features and advantages of the present invention will be made apparent from the following detailed description and the drawings.

### BRIEF DESCRIPTION OF THE DRAWINGS

The drawings illustrate embodiments presently contemplated for carrying out embodiments of the invention.

In the drawings:

FIG. 1 is a schematic of an imaging system according to an embodiment of the invention.

FIG. 2 is a flowchart setting forth the process of strain imaging according to an embodiment of the invention.

FIG. 3 is a scatter plot of strain data generated according to an embodiment of the invention.

FIG. 4 is a flowchart setting forth the process of histogram scaling according to an embodiment of the invention.

FIG. 5 is a cumulative histogram of strain data generated according to an embodiment of the invention.

### DETAILED DESCRIPTION OF THE INVENTION

Embodiments of the invention are directed to a system and method for strain imaging of a material. Strain data corresponding to a plurality of image frames is correlated to scale the data between frames and to decrease noise and artifacts. Each of the plurality of image frames corresponds to a distinct state of compression of the material.

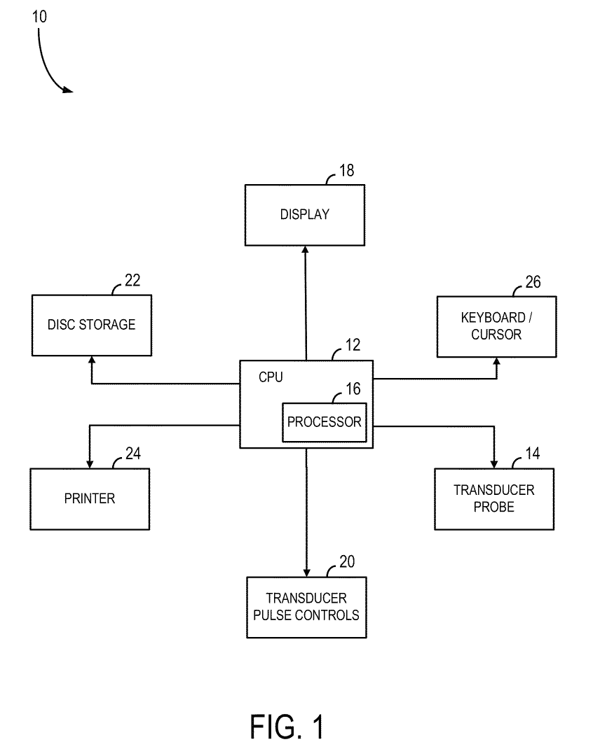

FIG. 1 illustrates a schematic diagram of an ultrasound imaging system 10 incorporating an embodiment of the invention. The ultrasound system or machine 10 includes a computer (i.e., central processing unit) 12 that is operationally connected to a transducer probe 14 that generates and receives ultrasonic sound waves. Computer 12 includes one or more processors 16. In accordance with conventional construction, the transducer probe generates and receives sound waves using piezoelectric crystals, cMUTs, pMUTs, PVDF, single crystal or another material known in the art that, when energized by an electric current, change shape rapidly so as to produce sound waves that travel outwardly to an object to be imaged. Conversely, when sound or pressure waves hit the piezoelectric crystals, the crystals emit electrical currents that can be processed by the computer and reconstructed to form an image that is displayed on computer monitor or display 18. Ultrasound system 10 further includes transducer pulse controls 20 that allow the operator to set and change the frequency and duration of the ultrasound pulses as well as the scan mode of the ultrasound machine. The commands from the operator are translated into changing electric currents that are applied to the transducer piezoelectric elements.

Computer 12 sends electrical currents to the transducer probe to emit sound waves and also receives the electrical pulses from the probes that were created from the returning echoes. Computer 12 also performs the necessary calculations involved in the processing of the received data for image reconstruction as well as other measurements that are carried out. Once the raw data is processed, computer 12 forms the image on monitor 18. Computer 12 may also store the processed data and/or image on a disc 22 or cause a copy of the image to be printed on printer 24. Computer 12 also communicates with a keyboard/cursor 26 which operates as an input device to allow the operator to add notes and to take measurements from the data.

As will be described in greater detail below, computer 12 of ultrasound system 10 is further configured to analyze the acquired ultrasound data for purposes of strain imaging. That is, computer 12 analyzes ultrasound data received from a biological or non-biological elastic tissue or material at differing states of compression. Monitor 18 can display B-mode images of the tissue with maps of the estimated strain data overlaid thereon. Additionally, such maps can be displayed alone, printed on printer 24, and/or stored on disc 22.

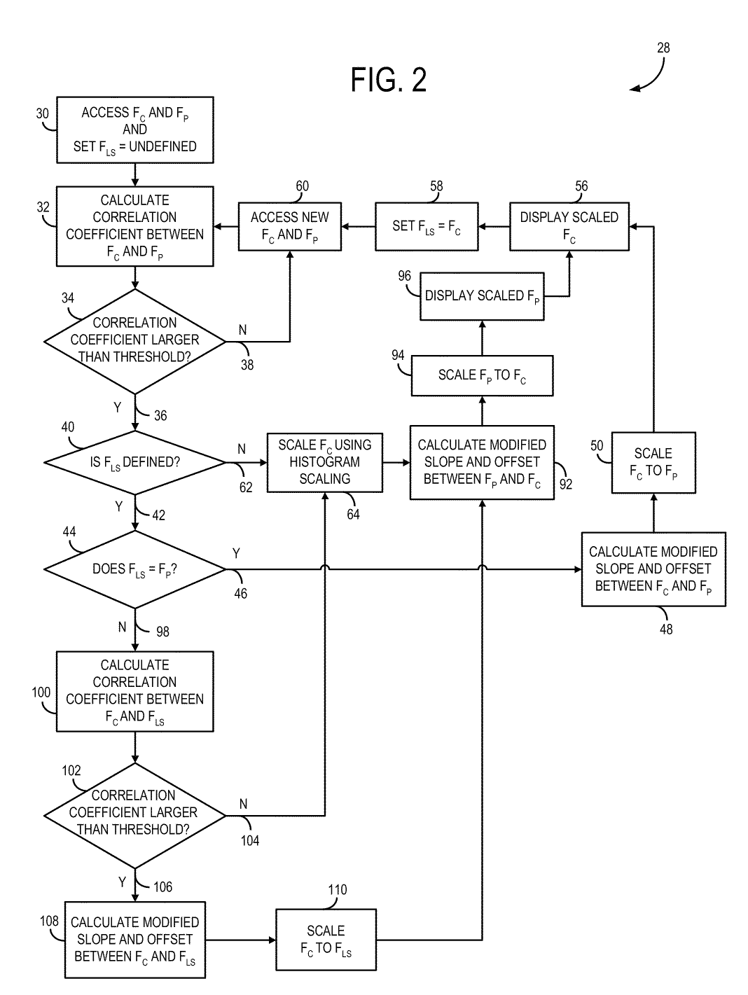

Referring now to FIG. 2, in one embodiment, one or more processors of a computer, such as computer 12 of FIG. 1, are programmed to carry out a technique 28 to scale unscaled strain data acquired from a strain imaging procedure. As used herein, the term unscaled strain data denotes a set of strain estimates made with an unknown applied stress. Technique 28 steps through pairs of adjacent (i.e., consecutively acquired) frames of estimated strain data to determine if the data in the two frames are well-correlated, using a method which is insensitive to the unknown scale factors in the two frames of strain data. If the data in two adjacent frames are well-correlated, technique 28 scales the strain data using a histogram scaling algorithm, a correlation scaling algorithm, or a combination thereof. If the data in adjacent frames are not well-correlated, technique 28 assumes that one or both of the frames contain non-useful data and does not perform any scaling.

Technique 28 begins at STEP 30 by accessing a first or current frame, F_{C}, and a second or previous frame, F_{P}, of a pair of consecutive frames of strain data. Technique 28 may either access stored frame data from a storage database or access real-time image data from an imaging system, such as, for example, ultrasound system 10 (FIG. 1), and estimate strain data therefrom. At STEP 30, technique 28 also sets the value of a previously scaled frame or last scaled frame, F_{LS}, which corresponds to the last frame of strain data which has been scaled, to a value representing an undefined value.

Next, technique 28 determines a correlation coefficient between the strain data in frames F_{C} and F_{P} at STEP 32. The correlation coefficient is a quantitative measure of the linear correlation between the strain data of frame F_{C} and the strain data of frame F_{P}. When STEP 32 is performed at some iterations of technique 28, the strain data in frame F_{P} may be unscaled strain data. When STEP 32 is performed at other iterations of technique 28, the strain data in frame F_{P} may be strain data which has been scaled by technique 28 in a previous iteration.

In one embodiment, the numerical representation of the correlation between frames of strain data is the Pearson correlation coefficient, which provides a measure of the linear relationship between the set of values Aᵢ and Rᵢ, where Aᵢ represents the strain values for frame F_{C} at positions i and Rᵢ represents the strain values for frame F_{P} at the same positions i. In one embodiment, the positions i in one or both frames may be modified to account for effects such as subject or transducer motion between frames, or for the estimated tissue displacement due to the applied stress. Specifically, the Pearson correlation coefficient, corr_coef may be determined by the equation:

$$corr\_coef = \frac{Σ ( {A}_{i} - 〈 A 〉 ) ( {R}_{i} - 〈 R 〉 )}{\sqrt{\sum {( {A}_{i} - 〈 A 〉 )}^{2} \sum {( {R}_{i} - 〈 R 〉 )}^{2}}} , \qquad ( Eqn . 1 )$$

where the sums are over the positions i and where [symbol] (A) [symbol] and [symbol] (R) [symbol] respectively represent the mean of the values Aᵢ and Rᵢ over the positions i. The Pearson correlation coefficient lies in the interval −1 to +1. Thus, a correlation coefficient of −1 and +1 indicates perfect negative or positive correlation, respectively, between Aᵢ and Rᵢ; a correlation coefficient of 0 indicates no correlation. In one embodiment, the correlation coefficient is determined using a subset of the strain data in a spatial region of interest (ROI). The ROI can be chosen as a spatial region in which the strain data is likely to be more accurate, such as, for example, the central region of the ultrasound frame.

At STEP 34, technique 28 compares the absolute value of the correlation coefficient, corr_coef with a correlation threshold value. According to one embodiment, the correlation threshold value is 0.7. If the absolute value of the correlation coefficient is larger than the threshold value 36, technique 28 assumes the strain data in frames F_{C} and F_{P} are well-correlated and scales frame F_{C} using a correlation scaling algorithm, a histogram scaling algorithm, or a combination thereof, as described in detail below. However, if the absolute value of the correlation coefficient is not greater than the threshold value 38, technique 28 assumes the strain data in frames F_{C} and F_{P} are poorly correlated, frame F_{C} is not scaled at this iteration, and technique 28 accesses new pairs of consecutive frames F_{C} and F_{P} at STEP 60 as described below. If technique 28 determines at STEP 34 that the absolute value of the correlation coefficient is larger than the threshold value 36, then at STEP 40, technique 28 determines whether any previous frame has been scaled by checking whether the value of frame F_{LS} is defined. If frame F_{LS} is defined 42, technique 28 determines if frame F_{P} is the last scaled frame F_{LS} at STEP 44. If frame F_{LS} is frame F_{P} 46, technique 28 proceeds to STEP 48.

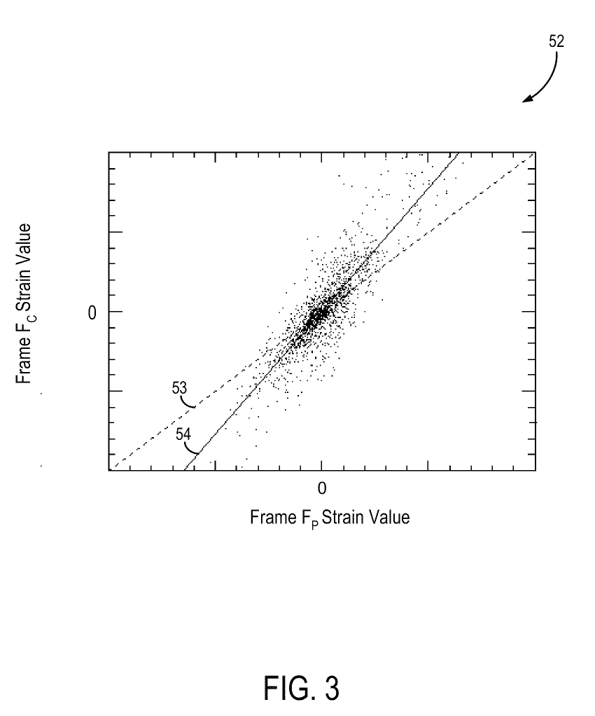

At STEPS 48 and 50, technique 28 performs a correlation scaling technique to scale the unscaled strain data in frame F_{C} to the scaled strain data in frame F_{P}. To determine the scaling factor, technique 28 first generates a scatter plot of strain data corresponding to frames F_{C} and Fₚ. As shown in FIG. 3, strain data from frames F_{C} and F_{P} are plotted on a scatter plot 52, with each plotted point corresponding to the two strain values estimated at the same position in the ultrasound image. The x-coordinate of a given plotted point is the strain value in one frame; the y-coordinate of the point is the strain value at the same position in the other frame. While the x-axis and y-axis of FIG. 3 are illustrated as corresponding to frame F_{C} and frame F_{P}, respectively, the axis to which each frame is assigned is arbitrary; the choice merely affects the interpretation of the scaling factor which is derived from the scatter plot. It is understood that only the x- and y-coordinate value pairing is needed by technique 28; the actual scatter plot, such as that shown in FIG. 3, need not be generated.

Referring to FIGS. 2 and 3, if the strain values in frames F_{C} and F_{P} differ solely by a scale factor, then the plotted points in scatter plot 52 will lie along a line, since the x- and y-coordinates of any point in scatter plot 52 differ only by the scale factor. If the scale factor is unity, than the plotted points will lie along a line 53 having a unity slope and a zero y-intercept. If the scale factor is a value other than unity, the plotted points may cluster around a line with non-unity slope. At STEP 48, the points in scatter plot 52 are fit to a line 54 to determine the slope and offset of a least-squares linear fit to the strain data in frame F_{C} as a function of the strain data in frame F_{P}. Specifically, a least-squares fit to line 54 having the equation:

Aᵢ=offset+slope·Rᵢ (Eqn. 2)

is performed on the strain data to determine the offset and slope of line 54, where Aᵢ represents the strain values in the ROI for frame F_{C} and Rᵢ represents the strain values in the ROI for frame F_{P}. As in the case of the calculation of the correlation coefficient, the least-squares fit may be calculated using strain data from a ROI for which the strain data is more likely to be reliable, such as, for example, the central region of the ultrasound frame. In one embodiment, the least-squares fit to line 54 may be calculated assuming the offset value is zero.

Based on the offset and slope values, a modified offset value, offset_mod, and a modified slope value, slope_mod, are calculated at STEP 48 using the following equations:

slope_mod=wt·slope+(1−wt)·unity_slope (Eqn. 3)

offset_mod=wt·offset (Eqn. 4),

where wt=(corr_coef)² and where unity_slope=1 when corr_coef is positive and unity_slope=−1 when corr_coef is negative. The modifications to the offset and slope values bias the offset toward zero and the slope of the linear fit toward unity (with the correct sign) as the magnitude of the correlation coefficient decreases from unity to the threshold value (i.e., as the confidence in the fitted slope and offset decreases). It is contemplated that alternative modifications to the offset and slope, other than Eqns. 3 and 4, and other functional forms for the weighting factor may be used. Alternatively, the modifications may be omitted, depending upon empirically observed usefulness of the modifications.

Finally, a scaled strain value, A_scaled, is calculated at STEP 50 for each strain data point of frame F_{C} using:

A_scaledᵢ=(Aᵢ−offset_mod)/slope_mod (Eqn. 5),

thereby scaling frame F_{C} to frame F_{P}. Eqn. 5 removes the observed slope and offset from the assumed linear form, Eqn. 2. By scaling frame F_{C} using the slope and offset of the linear fit, the correlation scaling algorithm minimizes the effect of the unknown difference in stress between two consecutive frames.

Still referring to FIG. 2, scaled frame F_{C} is displayed at STEP 56. At STEP 58, frame F_{C} is identified as the last scaled frame F_{LS}. Technique 28 then accesses a new pair of consecutive frames as frame F_{C} and F_{P} at STEP 60. To access the new pair of frames, technique 28 steps forward in time one frame such that the new pair of consecutive frames comprises frame F_{C} and the frame immediately succeeding it, relabeled as F_{P} and F_{C}, respectively, At STEP 32, a correlation coefficient between the new pair of frames F_{C} and F_{P} is calculated and technique 28 continues as described above.

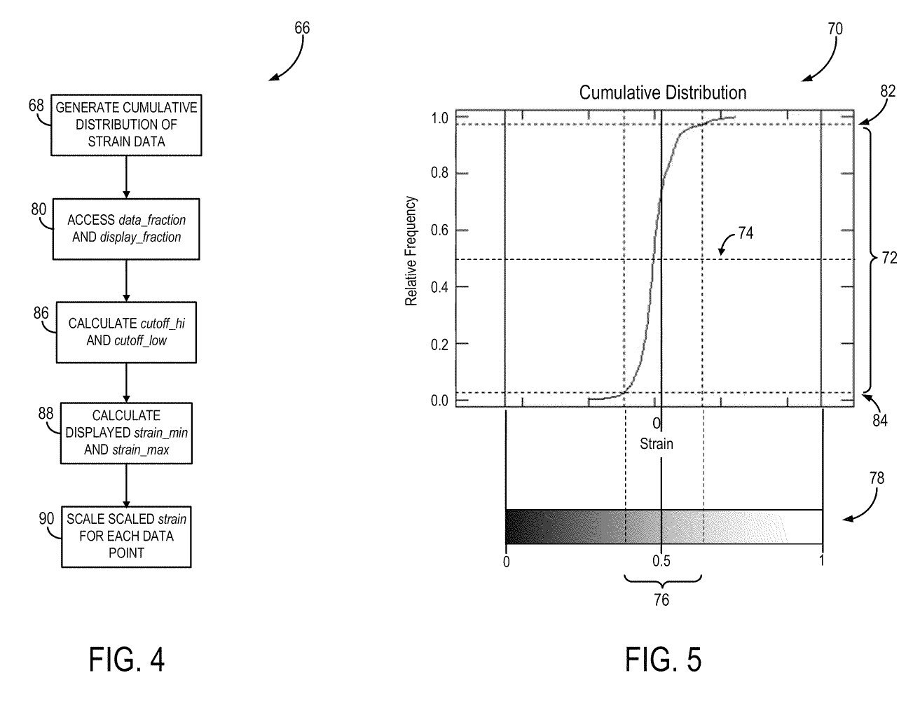

Referring back to STEP 40, if F_{LS} is not defined 62 (i.e., if no frames have been scaled thus far) then technique 28 scales frame F_{C} using a histogram scaling algorithm at STEP 64, as described below with respect to FIGS. 4 and 5.

The histogram algorithm scales a single frame of strain data without reference to another frame of strain data by calculating a linear function which maps the strain data to desired display values. Normalized display values are typically assumed to span the range from zero to one. Commonly, a normalized display value of zero represents the black level on a graylevel display, and a normalized display value of one represents the white level. The histogram scaling method is most useful when the data to be mapped is distributed rather compactly about a central value. The linear function is chosen so that most of the data values are mapped to a desired subset of the display range centered at the middle of the display range.

Referring to FIGS. 4 and 5, a histogram scaling algorithm 66 first generates a cumulative histogram using the unscaled strain data of frame F_{C} at STEP 68. In one embodiment, the strain data corresponding to an ROI centered in the image are used for the histogram scaling. The cumulative histogram is normalized to unity so that a histogram plot 70 of the strain data spans the range from zero to one. Thus, the cumulative histogram of frame F_{C} approximates the cumulative distribution of strain values for that frame. A central portion or data_fraction 72 of the strain values are chosen centered symmetrically about a central strain data value 74, such as, for example, the median value, 0.5. The strain data falling within data_fraction 72 are scaled to fall within a central portion or display_fraction 76 of a set of normalized display values 78. In other words, the histogram algorithm scales the strain data such that a selected central subset of the data occupy a selected central portion of the display values.

At STEP 80, histogram algorithm 66 accesses a pre-selected value of both data_fraction 72 and display_fraction 76. In one embodiment, data_fraction 72 and display_fraction 76 may be, for example, 0.95 and 0.25, respectively. Based on a selected value of data_fraction 72, an upper bound or cutoff_hi 82 and a lower bound or cutoff_lo 84 for the cumulative distribution strain data are calculated at STEP 86 using:

cutoff_lo=(1−data_fraction)/2 (Eqn. 5)

cutoff_hi=(1+data_fraction)/2 (Eqn. 6).

Eqns. 5 and 6 use a central strain data value 74 of 0.5. Using the selected display_fraction 76, a minimum displayed strain value or strain_min and a maximum displayed strain value or strain_max are calculated at STEP 88 by:

strain_max=data_mid+(data_hi−data_lo)/2 display_fraction) (Eqn. 7)

strain_min=data_mid−(data_hi−data_lo)/(2 display_fraction) (Eqn. 8),

where data_mid=(data_lo+data_hi)/2 and data_lo and data_hi are the data values corresponding to the values cutoff_lo and cutoff_hi in the cumulative distribution, respectively.

Once strain_min and strain_max are calculated, histogram algorithm 66 scales each strain data value or strain in the frame to the normalized display value or display_value at STEP 90 using:

display_value=(strain-strain_min)/(strain_max-strain_min) (Eqn. 9).

If the calculated display_value is less than zero, then display_value is set to zero. If the calculated display_value is greater than one, then display_value is set to one.

Referring again to FIG. 2, after frame F_{C} is scaled using histogram algorithm 66 at STEP 64, technique 28 scales frame F_{P} to the histogram-scaled frame F_{C} at STEPS 92 and 94. Technique 28 displays scaled frame F_{P} at STEP 96 and displays scaled frame F_{C} at STEP 56. Next, technique 28 proceeds to set frame F_{LS} equal to frame F_{C} at STEP 58 and accesses a new pair of frames F_{C} and F_{P} at STEP 60.

Referring back to STEP 44, if the last scaled frame, frame F_{LS}, is not frame F_{P} 98, technique 28 calculates a correlation coefficient between frames F_{C} and F_{LS} at STEP 100 using the Pearson correlation coefficient calculation described with respect to STEP 32. At STEP 102, if the correlation coefficient between frames F_{C} and F_{LS} is not larger than a correlation coefficient threshold value 104, technique 28 determines that the strain data in the two frames are not well-correlated and scales frame F_{C} using the histogram scaling algorithm at STEP 64, as described with respect to FIGS. 4 and 5. If the correlation coefficient between frames F_{C} and F_{LS} is larger than the threshold value 106, a modified slope and offset are calculated at STEP 108 and frame F_{C} is scaled to frame F_{LS} at STEP 110 using the correlation scaling algorithm described above with respect to FIG. 3. Technique 28 then scales frame F_{P} to frame F_{C} at STEPS 92 and 94. Frames F_{P} and F_{C} are displayed at STEP 96 and STEP 56, respectively. Technique 28 proceeds to set frame F_{LS} equal to frame F_{C} and access a new pair of frames F_{C} and F_{S} at STEPS 58 and 60 as described above.

Throughout technique 28, only scaled frames are displayed to a user. The missing frames may be interpolated or replaced by duplicated frames using methods well-known to those skilled in the art. Furthermore, according to one embodiment of the invention, the displayed scaled strain data may lag the real-time image acquisition by one or more frames. Alternatively, the scaled strain data may be stored for post-procedure analysis and/or display.

While an ultrasound imaging system is set forth above, it is contemplated that embodiments of the invention may be directed to any type of imaging system capable of acquiring image data at different states of compression of a material, such as, for example, an MR imaging system. Further, embodiments of the invention are equally applicable to live imaging as well as to positional and strain images acquired from an image storage database.

Therefore, in accordance with one embodiment of the present invention, a computer readable storage medium has a computer program stored thereon, which includes a set of instructions that when executed by a computer causes the computer to access positional data acquired from a material at a plurality of states of compression. The set of instructions also causes the computer to estimate a plurality of strain data sets from the positional data, each of the plurality of strain data sets corresponding to a different state of compression, and compare a first pair of strain data sets of the plurality of strain data sets with each other. The set of set of instructions further causes the computer to calculate a first measure of correlation from the comparison, scale a first strain data set of the first pair of strain data sets if the first measure of correlation is above a correlation threshold, and display the scaled first strain data set to a user.

In accordance with another embodiment of the present invention, a method includes accessing a first set of positional data, accessing a second set of positional data, and estimating a first set of strain data from the first set of positional data. The method also includes estimating a second set of strain data from the second set of positional data and comparing the first set of strain data with the second set of strain data. The method also includes calculating a first measure of correlation from the comparison, scaling the first set of strain data if the first measure of correlation is above a correlation threshold, and displaying the scaled first set of strain data to a user.

In accordance with yet another embodiment of the present invention, a system includes an imaging device configured to acquire a plurality of positional data sets, each positional data set comprising positional data of a material at a respective state of compression and a computer. The computer includes one or more processors programmed to access the plurality of positional data sets, estimate a first set of strain data from a first positional data set of the plurality of positional data sets, and estimate a second set of strain data from a second positional data set of the plurality of positional data sets. The one or more processors are further programmed to compare the first set of strain data with the second set of strain data, determine a first measure of correlation based on the comparison, scale the first set of strain data if the first measure of correlation is above a correlation threshold, and display an image of the scaled first set of strain data on a display.

A technical contribution for the disclosed system and method is that it provides for a computer-implemented method for strain imaging of a material.

The present invention has been described in terms of the preferred embodiment, and it is recognized that equivalents, alternatives, and modifications, aside from those expressly stated, are possible and within the scope of the appending claims.

What is claimed is:

1. A computer readable storage medium having a computer program stored thereon, the computer program comprising a set of instructions that when executed by a computer causes the computer to:
access positional data acquired from a material at a plurality of states of compression;
estimate a plurality of strain data sets from the positional data, each of the plurality of strain data sets corresponding to a different state of compression;
compare a first pair of strain data sets of the plurality of strain data sets with each other;
calculate a first measure of correlation from the comparison;
scale a first strain data set of the first pair of strain data sets if the first measure of correlation is above a correlation threshold; and
display the scaled first strain data set to a user.

2. The computer readable storage medium of claim 1 wherein the set of instructions further cause the computer to identify the first strain data set as a last-scaled data set.

3. The computer readable storage medium of claim 1 wherein the set of instructions further cause the computer to scale the first strain data set to a second strain data set of the first pair of strain data sets using a correlation algorithm if the second strain data set is a scaled data set, wherein the correlation algorithm causes the computer to:
generate a pairwise association of data from the first and second strain data sets;
generate an approximate linear fit to the pairwise association of data;
determine a slope and an offset from the linear fit; and
scale the first strain data set based on the slope and the offset.

4. The computer readable storage medium of claim 3 wherein the set of instructions that cause the computer to compare a first pair of strain data sets cause the computer to compare a first pair of strain data sets corresponding to a pair of consecutively acquired frames of positional data.

5. The computer readable storage medium of claim 1 wherein the set of instructions further cause the computer to:
scale the first strain data set using a histogram algorithm to produce a histogram-scaled first data set if the second strain data set is not scaled, wherein the histogram algorithm causes the computer to:
generate a cumulative distribution histogram from the first strain data set;
select a subset of the cumulative distribution histogram;
select a subset of a display range; and
scale the first strain data set based on an upper bound and a lower bound of the subset of the cumulative distribution histogram and an upper bound and a lower bound of the subset of the display range; and
scale the second strain data set to the histogram-scaled first strain data set using a correlation algorithm, wherein the correlation algorithm causes the computer to:
generate a pairwise association of data from the first and second strain data sets;
generate an approximate linear fit to the pairwise association of data;
determine a slope and an offset from the linear fit; and
scale the second strain data set based on the slope and the offset.

6. The computer readable storage medium of claim 1 wherein the set of instructions further cause the computer to:
compare a second pair of strain data sets from the plurality of strain data sets with each other, wherein the second pair of strain data sets includes the first strain data set and a last-scaled strain data set;
calculate a second measure of correlation from the comparison;
scale the first strain data set using a first correlation algorithm if the second measure of correlation is above the correlation threshold, wherein the first correlation algorithm causes the computer to:
generate a first pairwise association of data from the first strain data set and the last-scaled strain data set;
generate a first approximate linear fit to the first pairwise association of data;
determine a first slope and a first offset from the first linear fit; and
scale the first strain data set based on the first slope and the first offset; and
scale the second strain data set to the first strain data set using a second correlation algorithm, wherein the second correlation algorithm causes the computer to:
generate a second pairwise association of data from the first strain data set and the second strain data set;
generate a second approximate linear fit to the second pairwise association of data;
determine a second slope and a second offset from the second linear fit; and
scale the second strain data set based on the second slope and the second offset.

7. The computer readable storage medium of claim 1 wherein the set of instructions further cause the computer to:
compare a second pair of strain data sets from the plurality of strain data sets with each other, wherein the second pair of strain data sets includes the first strain data set and a last-scaled strain data set;
calculate a second measure of correlation from the comparison;
scale the first strain data set using a histogram algorithm if the second measure of correlation is below the correlation threshold, wherein the histogram algorithm causes the computer to:
generate a cumulative distribution histogram from the first strain data set;
select a subset of the cumulative distribution histogram;
select a subset of a display range; and
scale the first strain data set based on an upper bound and a lower bound of the subset of the cumulative distribution histogram and an upper bound and a lower bound of the subset of the display range; and
scale the second strain data set to the first strain data set using a correlation algorithm, wherein the correlation algorithm causes the computer to:
generate a pairwise association of data from the first strain data set and the second strain data set;
generate an approximate linear fit to the pairwise association of data;
determine a slope and an offset from the linear fit; and
scale the second strain data set based on the slope and the offset.

8. A method comprising:
accessing a first set of positional data;
accessing a second set of positional data;
estimating a first set of strain data from the first set of positional data;
estimating a second set of strain data from the second set of positional data;
comparing the first set of strain data with the second set of strain data;
calculating a first measure of correlation from the comparison;
scaling the first set of strain data if the first measure of correlation is above a correlation threshold; and
displaying the scaled first set of strain data to a user.

9. The method of claim 8 further comprising:
accessing a previously scaled set of strain data;
comparing the previously scaled set of strain data with the first set of strain data;
calculating a second measure of correlation from the comparison;
scaling the first set of strain data to the previously scaled set of strain data;
scaling the second set of strain data to the scaled first set of strain data; and
displaying the scaled second set of strain data to a user.

10. The method of claim 9 further comprising:
displaying a base image corresponding to the set of positional data; and
overlaying the base image with the displayed scaled set of strain data.

11. The method of claim 10 further comprising acquiring the first and second sets of positional data, wherein the first set of positional data corresponds to a first compressional state of the material and the second set of positional data corresponds to a second compressional state of the material.

12. A system comprising:
an imaging device configured to acquire a plurality of positional data sets, each positional data set comprising positional data of a material at a respective state of compression; and
a computer comprising one or more processors programmed to:
access the plurality of positional data sets;
estimate a first set of strain data from a first positional data set of the plurality of positional data sets;
estimate a second set of strain data from a second positional data set of the plurality of positional data sets;
compare the first set of strain data with the second set of strain data;
determine a first measure of correlation based on the comparison;
scale the first set of strain data if the first measure of correlation is above a correlation threshold; and
display an image of the scaled first set of strain data on a display.

13. The system of claim 12 wherein the one or more processors is further programmed to estimate a third set of strain data from a third positional data set of the plurality of positional data sets.

14. The system of claim 12 wherein the correlation threshold is approximately 0.7.

15. The system of claim 12 wherein the one or more processors is further programmed to scale the first set of strain data to produce a scaled first set of strain data if a second measure of correlation is above the correlation threshold.

16. The system of claim 15 wherein the one or more processors is further programmed to perform a correlation scaling algorithm to scale the second set of strain data to the scaled first set of strain data.

17. The system of claim 15 wherein the one or more processors is further programmed to:
perform a histogram scaling algorithm to scale the first set of strain data if the second set of strain data is not scaled; and
perform the correlation scaling algorithm to scale the second set of strain data to the histogram-scaled first set of strain data.

18. The system of claim 15 wherein the one or more processors is further programmed to:
estimate a third set of strain data from a third positional data set of the plurality of positional data sets;
compare the first set of strain data to the third set of strain data;
determine a third measure of correlation based on the comparison;
perform the correlation scaling algorithm to scale the first set of strain data to the third set of strain data to produce a correlation-scaled first set of strain data if the second set of strain data is not scaled and the third measure of correlation is above the correlation threshold; and
perform the correlation scaling algorithm to scale the second set of strain data to the correlation-scaled first set of strain data.

19. The system of claim 18 wherein the one or more processors is further programmed to:
perform the histogram scaling algorithm to scale the first set of strain data to produce a histogram-scaled first set of strain data if the second set of strain data is not scaled and the third measure of correlation is below the correlation threshold; and
perform the correlation scaling algorithm to scale the second set of strain data to the histogram-scaled first set of strain data.

20. The system of claim 12 wherein the imaging device is further configured to acquire the plurality of positional data sets.

21. The system of claim 20 wherein the one or more processors is further programmed to:
display a base image of the material based, the base image corresponding to the plurality of positional data sets; and
overlay the image of the scaled first set of strain data on the base image.

22. The system of claim 20 wherein the imaging device comprises an ultrasound device.

23. The system of claim 12 wherein the material comprises biological tissue.
